(19) United States
(12) Patent Application Publication
(10) Pub. No.: US 2005/0149188 A1
(43) Pub. Date: Jul. 7, 2005

(54) Anterior spinal implant

(76) Inventors: Stephen D. Cook, Pass Christian, MS (US); Kirk J. Bailey, Blairstown, NJ (US); Patrick Rousseau, Hardwick, NJ (US)

Correspondence Address: HARNESS, DICKEY & PIERCE, P.L.C., P.O. BOX 828, BLOOMFIELD HILLS, MI, 48303, US

(21) Appl. No.: 10/503,452

(22) PCT Filed: Feb. 7, 2003
(86) PCT No.: PCT/US03/03743

Related U.S. Application Data
(60) Provisional application No. 60/355,418, filed on Feb. 7, 2002.

(51) Int. Cl. A61F 2/44

(52) U.S. Cl. 623/17.11

(57) ABSTRACT

An anterior spinal implant includes a superior end face, an inferior end face and a sidewall extending between the superior end face and the inferior end face. The superior end face and the inferior end face are convex. In one application, the superior and inferior end faces are convex. The sidewall includes a convexly curved leading portion, a convexly curved trailing portion and a pair of substantially planar side portions. The superior and inferior end faces transition in a sagittal plane from a first radius of curvature to a second radius of curvature. The first radius of curvature is smaller than the second radius of curvature. The apexes of the superior and inferior end faces are offset from a center of the implant.

### CROSS-REFERENCE TO RELATED APPLICATION

This application claims priority to a provisional patent application which has been assigned U.S. Ser. No. 60/355,418, filed Feb. 7, 2002.

### FIELD OF THE INVENTION

The present invention generally pertains to orthopedic surgical procedures. More particularly, the present invention pertains to anterior spinal implants.

### BACKGROUND OF THE INVENTION

The human spinal column includes more than twenty discrete bones. These bones are generally similar in shape. Despite their similar shape, however, they do vary substantially in size in accordance with their individual position along the spinal column. The bones are anatomically categorized as being members of one of three classifications: cervical, thoracic, or lumbar. The cervical portion of the spinal column, which comprises the top of the spine up to the base of the skull, includes the first seven vertebrae. The intermediate twelve bones are thoracic vertebrae. The remaining five bones are the lumbar vertebrae.

The anterior portion of the spine includes a set of generally cylindrically shaped bones stacked one on top of the other which are referred to as the vertebral bodies. The vertebral bodies are separated from one another by cartilage spacers referred to as intervertebral discs. The intervertebral discs normally maintain a disc height between adjacent vertebral bodies.

The spinal column is a highly complex structure which houses and protects critical elements of the nervous system. In spite of these complexities, the spinal column is a highly flexible structure, capable of a high degree of curvature and twist through a wide range motion. Genetic or developmental irregularities, trauma, chronic stress, tumors, and disease, however, can result in spinal pathologies which either limit this range of motion, or threaten the critical elements of the nervous system housed within the spinal column.

In various orthopedic surgical procedures, it is necessary to stabilize portions of a spinal column relative to one another. This need is typically a result of disease, damage or congenital deformation. For example, when one or more intervertebral disks of the spine degenerate due to trauma or disease, the spinal cord or emergent nerve can become compressed. This condition results in chronic and sometimes debilitating, neck, back, or peripheral pain.

One method of treatment for intervertebral disk degeneration involves surgical decompression of nerves, reestablishment of the normal gap between adjacent vertebral bodies, and maintenance of the normal gap with an implant secured to the spinal column. For example, posterior implants are attached to the back of the spinal column generally by coupling to the pedicles with screws, or through hooks which attach under the lamina. The implants generally include elongate support rod elements which are coupled to the screws or hooks to immobilize several sequential vertebrae, for example to hold them stable so that adjacent vertebral bodies may be fused with bone graft.

Another method for treatment of intervertebral disk degeneration, the normal gap between adjacent vertebral bodies is surgically re-established and maintained with a rigid spacer inserted between the bodies. The rigid spacer is filled with bone graft material to facilitate bony fusion of the two vertebral bodies. A successful fusion stabilizes the spine, reduces pressure on the spinal cord and nerve roots, and reduces or eliminates back pain.

Yet another method for treatment of intervertebral disk degeneration involves discectomy and introduction of an implant between two adjacent vertebral bodies. Such intervertebral implants re-establish the normal gap between adjacent vertebral bodies. In some known applications, the use of intervertebral implants maintains a degree of the natural movement permitted between adjacent vertebral bodies.

One method of treatment for intervertebral disk degeneration involves surgical decompression of nerves, reestablishment of the normal gap between adjacent vertebral bodies, and maintenance of the normal gap with an implant secured to the spinal column. For example, posterior implants are attached to the back of the spinal column generally by coupling to the pedicles with screws, or through hooks which attach under the lamina. The implants generally include elongate support rod elements which are coupled to the screws or hooks to immobilize several sequential vertebrae, for example to hold them stable so that adjacent vertebral bodies may be fused with bone graft.

While known devices for spinal stabilization have proven to be effective in various applications, they nevertheless can be the subject of certain improvements. In this regard, many known devices for spinal stabilization are associated with areas of localized stress on adjacent vertebral end plates and/or migration and retropulsion from proper positioning within the spinal column. There remains a need in the pertinent art to overcome these and other limitations associated with known devices.

### SUMMARY OF THE INVENTION

It is a general object of the present invention to provide an anterior spinal implant that resists migration and retropulsion from proper positioning within the spinal column.

It is another object of the present invention to provide an anterior spinal implant that provides increased contact area with adjacent vertebral end plates.

It is a related object of the present invention to provide an anterior spinal implant that disperses stresses over a larger area on adjacent vertebral end plates.

It is another object of the present invention to provide an anterior spinal implant that re-establishes the disk height and allows for articulation against the end plates of the adjacent vertebral bodies.

Further objects of the present invention include provision for an anterior spinal implant that provides less subsidence, improved cartilage survival, improved motion in the associate spinal segment and improved spinal stability.

Additional objects and features of the present invention will become apparent from the following description and appended claims, taken in conjunction with the accompanying drawings.

In one particular form, the present invention provides an anterior spinal implant including a superior end face, an inferior end face and a generally cylindrical sidewall. The generally cylindrical sidewall extends between the superior end face and the inferior end face. The superior end face and the inferior end face are convex.

In another form, the present invention provides an anterior spinal implant having a convexly shaped superior end face and a convexly shaped inferior end face. The anterior spinal implant additionally includes a sidewall extending between the superior end face and the inferior end face. The sidewall includes a convexly curved leading portion, a convexly curved trailing portion and a pair of substantially planar side portions.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a simplified lateral side view of a anterior spinal implant constructed in accordance with the teachings of a first preferred embodiment of the present invention, the anterior spinal implant shown associated with portions of a human spine.

FIG. 2 is a perspective view of the anterior spinal implant according to the first preferred embodiment of the present invention.

FIG. 3 is a lateral side view of the anterior spinal implant according to the first preferred embodiment of the present invention.

FIG. 4 is a superior end view of the anterior spinal implant according to the first preferred embodiment of the present invention.

FIG. 5 is a perspective view of an anterior spinal implant constructed in accordance with a second preferred embodiment of the present invention.

FIG. 6 is a lateral side view of the anterior spinal implant constructed in accordance with the teachings of the second preferred embodiment of the present invention.

FIG. 7 is a perspective view of an anterior spinal implant constructed in accordance with a third preferred embodiment of the present invention.

FIG. 8 is a lateral side view of the anterior spinal implant constructed in accordance with the teachings of the third preferred embodiment of the present invention.

FIG. 9 is a posterior end view of the anterior spinal implant constructed in accordance with the third preferred embodiment of the present invention.

FIG. 10 is a lateral side view of an anterior spinal implant constructed in accordance with the teachings of a fourth preferred embodiment of the present invention.

FIG. 11 is a lateral side view of the anterior spinal implant constructed in accordance with the teachings of the fourth preferred embodiment of the present invention.

FIG. 12 is a posterior end view of the anterior spinal implant constructed in accordance with the fourth preferred embodiment of the present invention.

FIG. 13 is a superior end view of the anterior spinal implant according to a fifth preferred embodiment of the present invention.

FIG. 14 is a lateral side view of the anterior spinal implant constructed in accordance with the teachings of the fifth preferred embodiment of the present invention.

FIG. 15 is a posterior end view of the anterior spinal implant constructed in accordance with the fifth preferred embodiment of the present invention.

FIG. 16 is a perspective view of a tool for implantation of the anterior spinal implant according to the fifth preferred embodiment of the present invention, the tool shown operatively associated with the implant of the fifth preferred embodiment.

### DETAILED DESCRIPTION OF THE PREFERRED EMBODIMENTS

The following description of the preferred embodiment(s) is merely exemplary in nature and is in no way intended to limit the invention, its application, or uses.

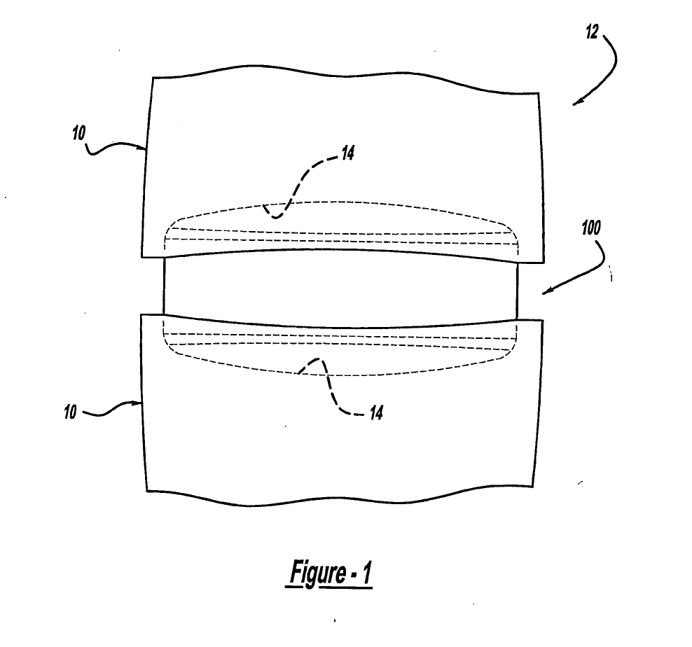

With initial reference to the simplified environmental view of FIG. 1, an anterior spinal implant constructed in accordance with the teachings of a first preferred embodiment of the present invention is illustrated and generally identified at reference character 100. The anterior spinal implant 100 is shown in the environmental view of FIG. 1 implanted between adjacent vertebral bodies 10 of a lumbar portion of a human spine 12. Those skilled in the art will readily appreciate that the application shown in FIG. 1 is merely exemplary. In this regard, the teachings of the present invention will be understood to be equally applicable for stabilizing other segments of the spinal column.

The anterior spinal implant is intended to abut the end plates 14 of the adjacent vertebral bodies 10. The remaining embodiments of the present invention are intended to be similarly situated within the human spine 12. One particular method of implantation will be described below.

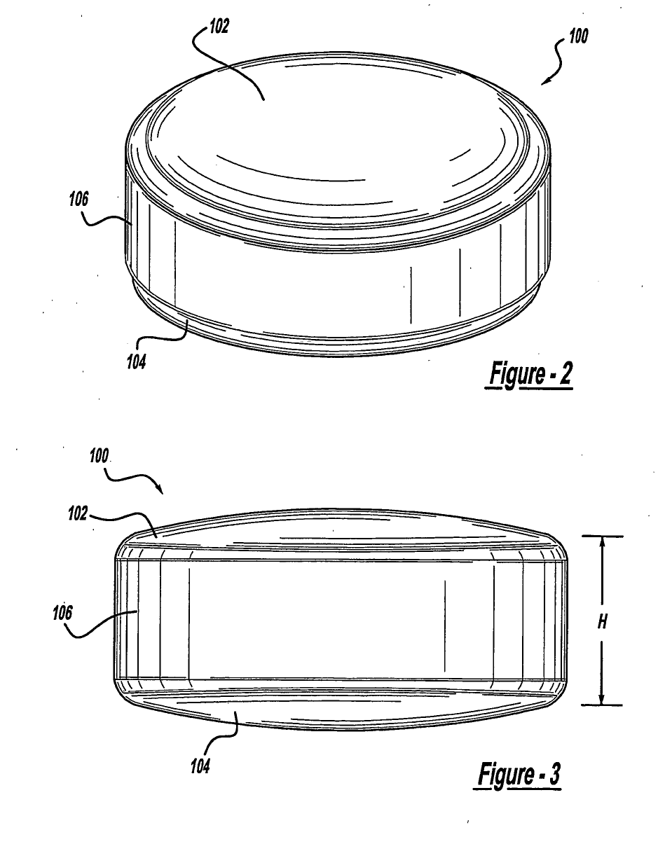

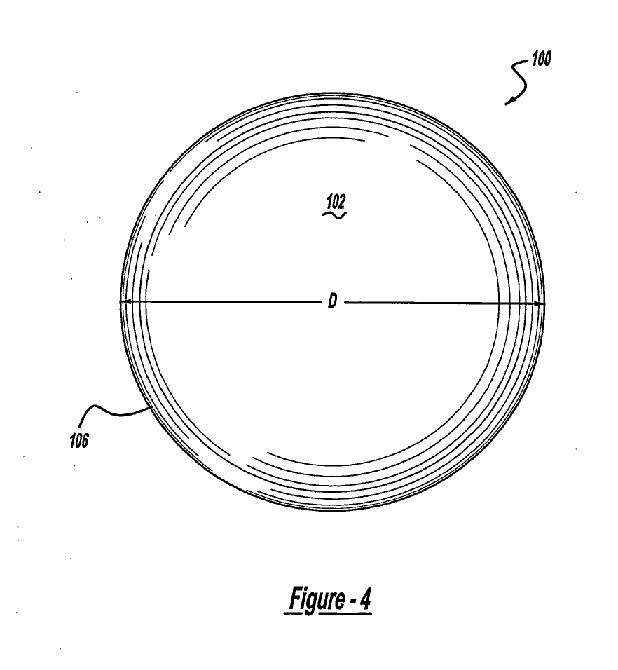

With continued reference to FIG. 1 and additional reference to FIGS. 2 through 4, the anterior spinal implant 100 will be described in greater detail. The anterior spinal implant 100 is illustrated to include a generally cylindrical shape having a superior face 102 and an inferior face 104. The superior face 102 and the inferior face 104 are preferably smooth and abut end plates 14 of the adjacent vertebral bodies 10. The superior face 102 and the inferior face 104 are connected by a generally cylindrical sidewall 106 by radiused corners 108.

The implant 100 has a diameter D and a constant height H. In certain preferred applications for the lumbar portion of the human spine, the diameter D ranges from approximately 16.5 mm to approximately 25 mm. In these applications, the height H ranges from approximately 8 mm to 20 mm. It will be understood that dimensions and relative dimensions of these preferred applications and any preferred applications discussed below in connection with other embodiments of the present invention are merely exemplary. In this regard, the teachings of the present invention apply to a much wider range of dimensions depend upon anatomical considerations and surgeon preferences.

As shown most particularly in FIG. 3, the superior face 102 and the inferior face 104 are preferably convex in shape. In the exemplary embodiment illustrated, the superior and inferior faces 102 and 104 are partially spherical in shape. In this regard, the superior face 102 includes a spherical cephalad radius and the inferior face 104 includes a spherical caudad radius. In the embodiment illustrated, the cephalad radius is substantially equal to the caudad radius. Preferably, the cephalad and caudad radii of curvature range from approximately 20 mm to approximately 25 mm.

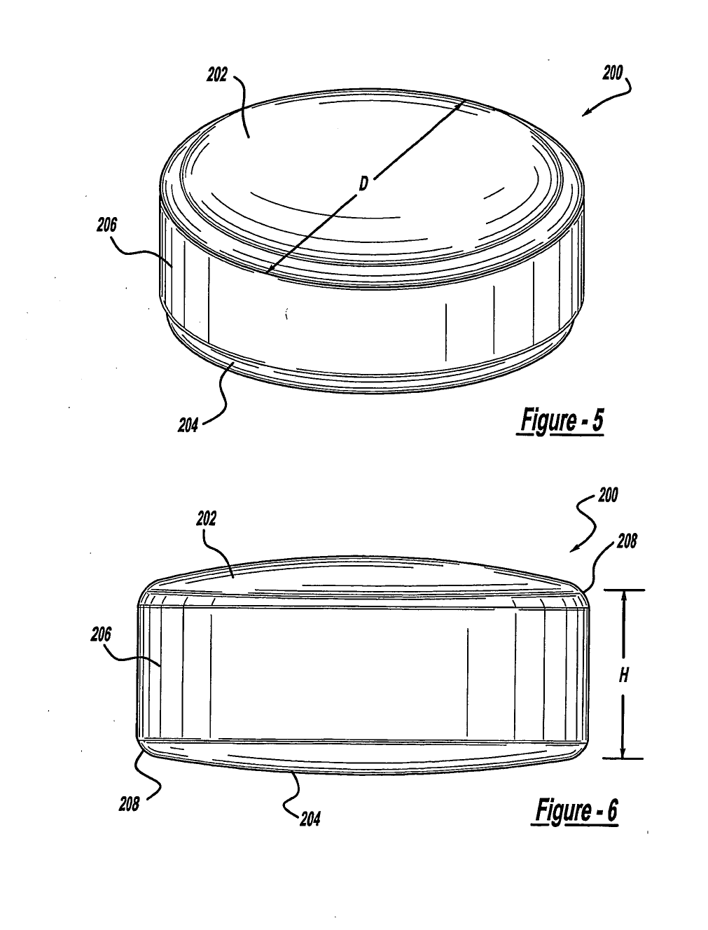

With reference to FIGS. 5 and 6, an anterior spinal implant constructed according to the teachings of a second preferred embodiment of the present invention is illustrated and generally identified at reference element 200. As with the spinal implant 100 of the first preferred embodiment, the spinal implant 200 is generally cylindrical in shape having a superior face 202 and an inferior face 204. The superior face 202 and inferior face 204 of the spinal implant 200 of the second preferred embodiment are smooth and abut end plates 14. The superior and inferior faces are connected by a generally cylindrical sidewall 206 through radiused corners 208.

In the embodiment illustrated, the cephalad and caudad radii of the superior and inferior end faces 202 and 204 are partially spherical (shown most clearly in FIG. 6). In contrast to the implant 100 of the first preferred embodiment, the cephalad and caudad radii are distinct. In the exemplary embodiment illustrated, the caudad radius is greater than the cephalad radius. Preferably, the caudad radius is approximately twice the cephalad radius. In one particular application, the cephalad radius is 25 mm and the caudad radius is 50 mm. In another particular application, the cephalad radius is 20 mm and the caudad radius is 40 mm. The remaining dimensions of the implant 200 are similar to corresponding dimensions of the implant 100.

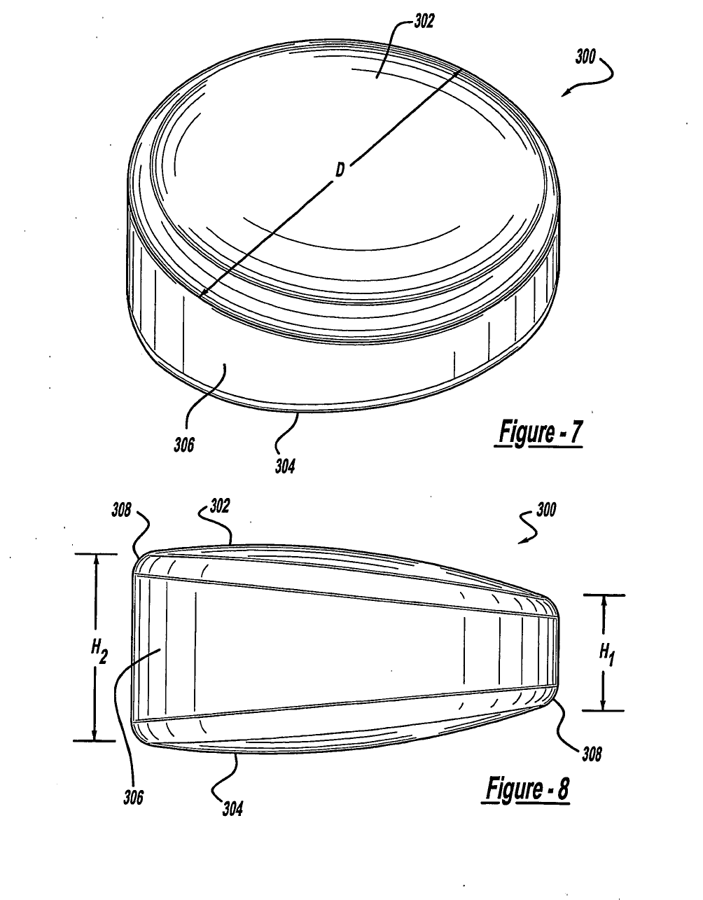

Turning to FIGS. 7 through 9, an anterior spinal implant constructed according to the teachings of a third preferred embodiment of the present invention is generally identified at reference character 300. As with the spinal implant 100 of the first preferred embodiment, the spinal implant 300 is generally cylindrical in shape having a superior face 302 and an inferior face 304. The superior face 302 and inferior face 304 of the spinal implant 300 of the third preferred embodiment are smooth and abut end plates 14. The superior and inferior faces 302 and 304 are connected by a generally cylindrical sidewall 306 through radiused corners 308.

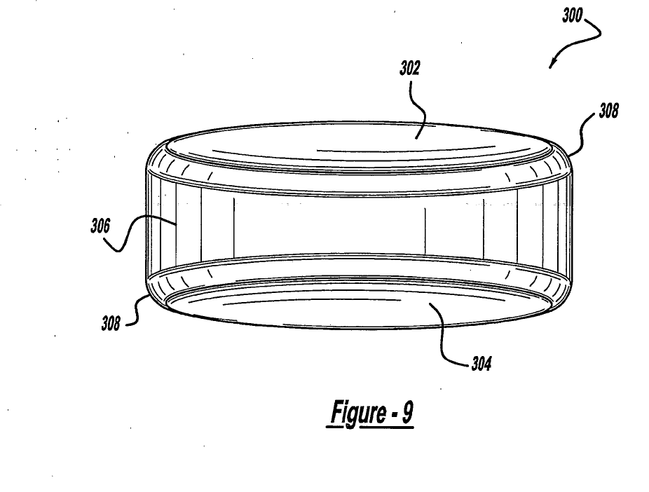

In the embodiment illustrated, the superior face 302 and the inferior face 304 are convex. In contrast to the first and second embodiments, the superior and inferior end faces 302 and 304 of the implant 300 include distinct sagittal and coronal radii. In one exemplary application, the sagittal radii of curvature (shown most clearly in FIG. 8) of the superior and inferior end faces 302 and 304 range from approximately 20 mm to approximately 25 mm. In this particular embodiment, the coronal radii of curvature ranges (shown most clearly in FIG. 9) from approximately 25 mm to approximately 40 mm.

The anterior spinal implant 300 further differs from the anterior spinal implants 100 and 200 in that the sidewall 306 has a variable height. As shown in FIG. 8, the spinal implant 300 has a first height H₁ adjacent an anterior end 302 and a second height H₂ adjacent a posterior end 304 that are distinct. The difference between the anterior height H₁ and the posterior height H₂ functions to shift the apexes posteriorly. This shift of the apexes allows the apexes of the implant 300 to be sealed in the nuclear recesses of the end plates 14. In certain preferred embodiments, the anterior height H₁ ranges from approximately 8 mm to approximately 20 mm and the cylindrical diameter D ranges from approximately 16.5 mm to approximately 25 mm. The remaining dimensions of the implant 300 are similar to corresponding dimensions of the implant 100.

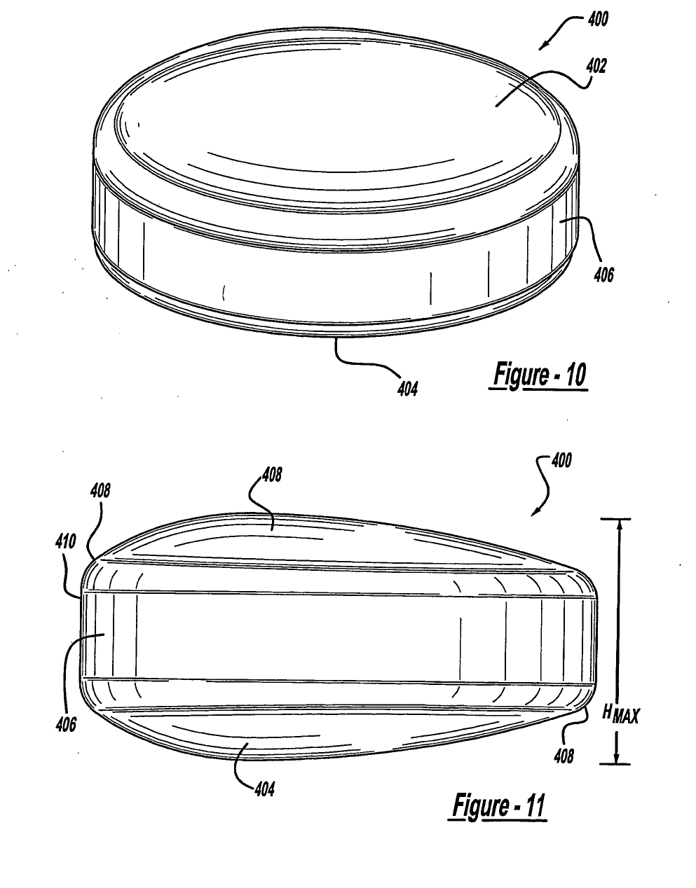

With reference to FIGS. 10 through 12, an anterior spinal implant constructed in accordance with the teachings of a fourth preferred embodiment of the present invention is illustrated and generally identified at reference number 400. As with the spinal implant 100 of the fourth preferred embodiment, the spinal implant 400 is generally cylindrical in shape having a superior face 402 and an inferior face 404. The superior face 402 and inferior face 404 of the spinal implant 400 of the fourth preferred embodiment are smooth and abut the end plates 14. The superior and inferior faces 402 and 404 are connected by a generally cylindrical sidewall 406 through radiused corners 408.

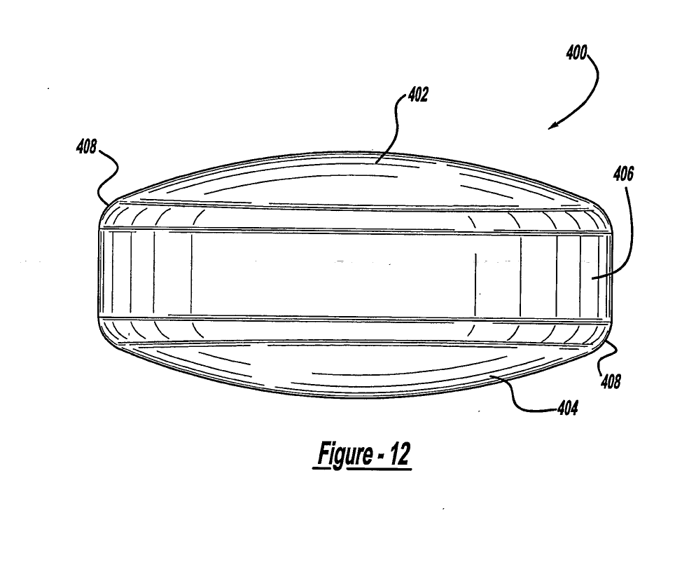

The anterior spinal implant 400 of the fourth preferred embodiment is similar to the implant 300 of the third preferred embodiment in that the superior and inferior end faces 402 and 404 of the implant 400 are distinct. The anterior spinal implant 400 differs from the implant 300 in that the apexes of the sagittal radii of the superior and inferior faces 402 and 404 are offset toward a posterior end of the implant 400. This offset is shown most clearly in FIG. 11. In the embodiment illustrated, the apex of the sagittal radii is preferably between approximately 7 mm and 10 mm from a posterior end 410 of the anterior spinal implant.

The anterior spinal implant 400 includes a maximum height Hₘₐₓ from the apex of the sagittal radius of the superior end face 402 to the apex of the sagittal radius of the inferior end face 404. The maximum height Hₘₐₓ ranges from approximately 8 mm to approximately 20 mm. Where the maximum height Hₘₐₓ is approximately 8 mm, the sagittal radii of the superior and inferior faces 102 and 104 is approximately 90 mm. Where the maximum height Hₘₐₓ is approximately 20 mm, the sagittal radii of the superior and inferior faces 402 and 404 is approximately 25 mm. The remaining dimensions of the implant 400 are similar to corresponding dimensions of the previously described embodiments.

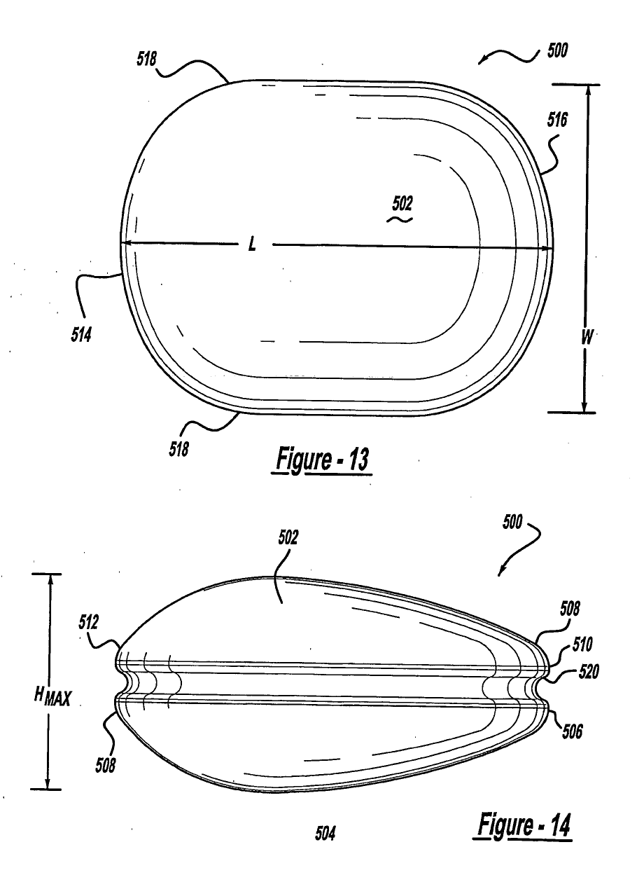

With reference to FIGS. 13 through 15, an anterior spinal implant constructed in accordance with the teachings of a fifth preferred embodiment of the present invention is illustrated and generally identified at reference number 500. As with the implants of the prior embodiments, the spinal implant 500 generally includes a superior face 502 and an inferior face 504 connected by a sidewall 506 through radiused corners 508. The superior face 502 and inferior face 504, which again are smooth and intended to abut the end plates 14, are convex. In the embodiment illustrated, the sagittal curvatures of the superior and inferior end faces 502 and 504 are identical. Further, both the superior and inferior faces 502 and 504 transition from a first or anterior radius of curvature adjacent an anterior end 510 of the implant 500 to a second or posterior radius of curvature adjacent a posterior end 512 of the implant 500.

In preferred applications, the sagittal radius of curvature of the superior face 502 adjacent the anterior end 510 is approximately 30 mm and the radius of curvature of the superior face 502 adjacent the posterior end 512 is approximately 10 mm to approximately 14 mm. In these particular applications, the radius of curvature of the inferior face 504 adjacent the anterior end 510 is approximately 30 mm and the radius of curvature of the inferior face 504 adjacent the posterior end 512 ranges from approximately 11 mm to approximately 13 mm.

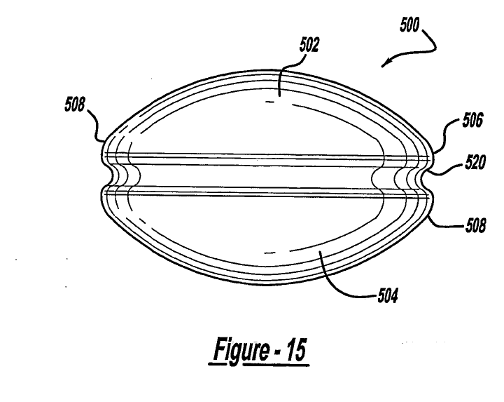

The apexes of the sagittal radii of the superior and inferior faces 502 and 504 are preferably offset. As shown most clearly in FIG. 14, the apexes are offset toward the posterior end 512 of the spinal implant 500. In the embodiment illustrated, the apexes are offset from the posterior end 512 between approximately 7 mm and approximately 10 mm depending on the height of the implant.

The radii of curvature of both the superior and inferior faces 502 and 504 in a coronal direction is variable from the anterior end 510 to the posterior end 512 of the implant. In the embodiment illustrated, the coronal radii of the superior face 502 and inferior face 504 through any particular cross section are substantially identical. In certain preferred applications, the coronal radii of curvature through the apexes of the superior face 502 and inferior face 504 ranges from approximately 17 mm to approximately 11.5 mm. Where the maximum height Hₘₐₓ is approximately 8 mm, the coronal radii of curvature are approximately 17 mm. Where the maximum height is approximately 16 mm, the coronal radii of curvature are approximately 11.5 mm.

Distinct from the prior described embodiments, the sidewall 506 of the implant 500 is not continuously curved. As perhaps shown most clearly in the superior view of FIG. 13, the sidewall includes a convex posterior side 514 and a convex anterior side 516. The sidewall is further shown to include a pair of generally planar lateral portions 518. The lateral side portions 518 further accommodate lateral bending of the spine 12. In this regard, the reduced geometry avoids potential pinching of the implant 500 between the vertebral bodies 10.

The implant 500 preferably has a width W between the lateral sides 518 of approximately 17 mm. The implant 500 additionally includes a length L between the posterior side 514 and the anterior side 516 which ranges between approximately 18 mm and 22 mm. The anterior spinal implant 500 includes a maximum height Hₘₐₓ from the apex of the superior end face 502 to the apex of the inferior end face 504. The maximum height Hₘₐₓ ranges from approximately 8 mm to approximately 16 mm.

The implant 500 of the fifth preferred embodiment of the present invention further differs from the prior described embodiments in that the sidewall 506 is formed to include a groove 520 that extends completely around the perimeter. The groove is intended to engage an implantation tool 600 in a manner to be described below. It will be understood that the other embodiments of the present invention may be similarly formed to include a groove for cooperation with an implantation tool.

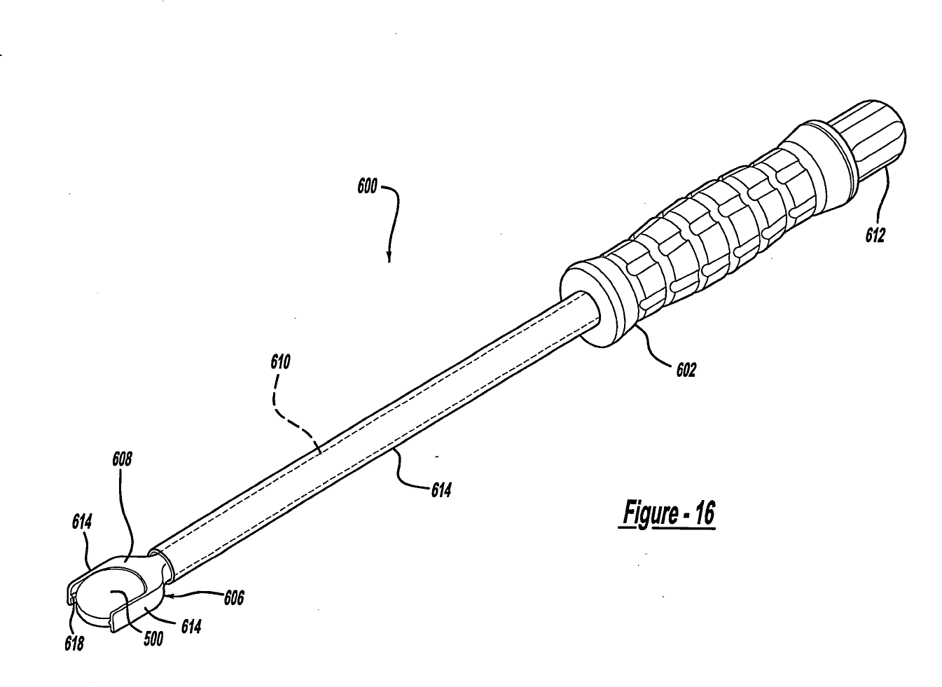

With particular reference to FIG. 16, one suitable tool 600 for implantation of the spinal implants of the present invention is illustrated. The insertion tool 600 is shown operatively associated with the spinal implant 500 of the fifth preferred embodiment of the present invention. The insertion tool is generally illustrated to include a handle 602, a sleeve 604 extending from the handle, and an engagement member 606 for releasably engaging the spinal implant 500. The sleeve 604 is a hollow cylinder. The engagement member includes a jaw 608 for directly engaging the implant and a shaft 610. The shaft extends through the sleeve 604 and is threadably engaged by a cap 612. In the preferred embodiment, the engagement member 606 is constructed of plastic and is disposable.

The jaw 608 of the engagement member includes a pair of spaced apart prongs 614 that define an opening for receiving the spinal implant 500. Preferably, the opening defined by the prongs is slightly smaller than the width of the spinal implant 500. In this manner, insertion of the spinal implant 500 into the opening functions to elastically deform the prongs 600. The resilient nature of the plastic retains the spinal implant 500 within the opening. In the preferred embodiment, the jaw 608 is formed to include a rail 618 that engages three sides of the spinal implant 500. Specifically, the rail 618 engages the groove 520 on the spinal implant 500 to maintain the orientation of the spinal implant relative to the jaw 608.

Depending on surgeon's preference, the anterior spinal implants of the present invention may be inserted from an anterior approach or a posterior approach. If the implant 500 is to be surgically implanted anteriorly, the implant 500 is positioned within the jaw 608 such that the posterior end 512 is the leading end. Conversely, if the implant 500 is to be surgically implanted posteriorly, the anterior end 510 of the spinal implant 500 will be the leading end. The posterior offset of the apexes of the superior and inferior end faces 502 and 504 allow the implant to be seated in the nuclear recesses of the end plates 14. In this manner, the fulcrum of the nuclear recess is restored and the natural function of the spine is substantially returned. The end plates 14 may articulate in a natural manner relative to the smooth end faces 502 and 504 of the implant 500.

Upon proper positioning of the implant 500, retraction of the spine 12 is released. The release causes the end plates 14 of the spine 12 to at least partially load the spinal implant 500. The load on the implant 500 is to a sufficient degree such that withdrawal of the insertion tool 600 causes the prong 614 to resiliently expand, thereby leaving the spinal implant 500 between the vertebral bodies 10.

Upon implantation, the various embodiments of the present invention resist migration and retropulsion from their proper positioning within the spinal column. The various embodiments also provide an increase contact area with the adjacent end plates and disperse stresses over a larger area on the end plates. The implants additionally provide less subsidence, improve cartilage survival, improve motion in the associated spinal segment and improve spinal stability.

The anterior spinal implants 100, 200, 300, 400, and 500 of the preferred embodiments present invention may be constructed of any material having suitable biocompatibility and strength characteristics. Suitable materials include, but are not limited to, cobalt chromium alloy and pyrolytic carbon. The various embodiments of the present invention may also be constructed of allograft bone.

The description of the invention is merely exemplary in nature and, thus, variations that do not depart from the gist of the invention are intended to be within the scope of the invention. Such variations are not to be regarded as a departure from the spirit and scope of the invention.

1. An anterior spinal implant comprising:
a superior end face;
an inferior end face; and
a sidewall extending between the superior end face and the inferior end face;
wherein the superior end face and the inferior end face are convex.

2. The anterior spinal implant of claim 1, wherein the superior end face is spherical and defined by a spherical cephalad radius and further wherein the interior end face is spherical and defined by a spherical caudad radius.

3. The anterior spinal implant of claim 2, wherein the cephalad radius is substantially identical to the caudad radius.

4. The anterior spinal implant of claim 3, wherein the cephalad radius ranges from approximately 20 mm to approximately 25 mm.

5. The anterior spinal implant of claim 2, wherein the cephalad radius ranges from approximately 20 mm to approximately 25 mm.

6. The anterior spinal implant of claim 2, wherein the cephalad radius and the caudad radius are distinct.

7. The anterior spinal implant of claim 6, wherein the caudad radius is greater than the cephalad radius.

8. The anterior spinal implant of claim 6, wherein the caudad radius is approximately twice the cephalad radius.

9. The anterior spinal implant of claim 8, wherein the cephalad radius ranges from approximately 20 mm to approximately 25 mm.

10. The anterior spinal implant of claim 1, further comprising an anterior height and a posterior height, the posterior height being greater than the anterior height.

11. The anterior spinal implant of claim 1, wherein an apex of at least one of the superior end face and inferior end face is posteriorly offset.

12. The anterior spinal implant of claim 11, wherein an apex of the superior end face and an apex of the inferior end face are posteriorly offset.

13. The anterior spinal implant of claim 1, wherein each of the superior end face and inferior end face includes a sagittal radius and a coronal radius, the sagittal and coronal radii being distinct.

14. The anterior spinal implant of claim 1, wherein each of the superior end face and inferior end face includes a sagittal radius with an apex, the apex being substantially closer to a posterior end of the implant than an anterior end of the implant.

15. The anterior spinal implant of claim 1, wherein the apex is between approximately 7 mm and 10 mm from the posterior end.

16. The anterior spinal implant of claim 1, wherein the sidewall is generally cylindrical.

17. An anterior spinal implant comprising:
a convexly shaped superior end face;
a convexly shaped inferior end face;
a sidewall extending between the superior end face and the inferior end face, the sidewall including a convexly curved leading portion, a convexly curved trailing portion and a pair of substantially planar side portions.

18. The anterior spinal implant of claim 17, wherein the superior end face and the inferior end face both transition from the leading portion to the trailing portion from a first radius of curvature to a second radius of curvature.

19. The anterior spinal implant of claim 18, wherein the second radius of curvature is substantially greater than the first radius of curvature.

20. The anterior spinal implant of claim 17, wherein an apex of at least one of the superior end face and inferior end face is posteriorly offset.
